(19) United States
(12) Patent Application Publication
(10) Pub. No.: US 2003/0230310 A1
(43) Pub. Date: Dec. 18, 2003

(54) Method and apparatus useful for reducing neuromusculoskeletal imbalance

(76) Inventor: John Brent Day, Boulder, CO (US)

Correspondence Address: David V. Radack, 600 Grant Street, Pittsburgh, PA, 15219, US

(21) Appl. No.: 10/166,658

(22) Filed: Jun. 12, 2002

(51) Int. Cl. A61F 5/00

(52) U.S. Cl. 128/845; 606/237

(57) ABSTRACT

There is provided a method of alleviating or reducing the symptoms of uneven pelvic rotation in a patient suffering therefrom, comprising: (a) selecting the patient; (b) identifying the patient's functionally short leg; (c) positioning the patient in a substantially prone position, thereby reducing any weight supported by the patient's pelvis; (d) while the patient is in the substantially prone position, simultaneously elevating a front portion of the patient's cephalic thigh adjacent the forwardly rotated acetabular joint and removing restrictions to the rearward rotation of said acetabular joint, while elevating a cephalic portion of the front region of the ilium on the other side; and, (e) repeating step (d) at least once per month. Therapeutic wedges useful in the method are also provided.

There is also provided a method of relieving or alleviating the symptoms of chronic spinal movement in the neck in a patient suffering therefrom, comprising: (a) selecting the patient; (b) identifying the direction of spinal movement/misalignment in the patient's neck; (c) positioning the patient in a substantially horizontal position looking upward so as to reduce any weight supported by the patient's neck; and, (d) applying a consistent force to the back of the patient's neck in the region of the second to fourth cervical vertebrae, thereby urging lateral displacement of the cervical spine in a direction opposite to the direction of spinal movement/misalignment in the patient's neck. Therapeutic forms such as neck rolls useful in the method are also provided.

### FIELD OF THE INVENTION

The invention relates generally to a method and apparatus for reducing undesired or excessive neuromusculoskeletal imbalance, and particularly imbalance in the lumbo-pelvic anatomy and cervical spine.

### BACKGROUND OF THE INVENTION

Skeletal and soft tissue imbalance may take several forms, including lumbo-pelvic imbalance and imbalance of the cervical spine. Actual differences in the length of the bones in the left and right legs of humans are relatively rare. However, skeletal and soft tissue imbalance frequently results in functional differences in leg length. This condition can lead to significant discomfort, including lower back and neck pain.

Lower back and neck pain cause significant discomfort for a large number of individuals, and have been implicated in high levels of absenteeism due to un-managed pain, as well as the extensive use of non-steroidal anti-inflammatories and other pain-reducing agents.

A variety of treatments for lower back and neck pain have been proposed, many of which involve contoured pillows which a patient can place under the affected area, to encourage an appropriate rear-front curvature of the spine, thereby possibly relieving stress and associated pain.

For example, Canadian Patent 2,172,304 of Gostine discloses a therapeutic pillow for use under the neck or lumbar spine, to encourage correct rear-front curvature. Similarly, pillows were also proposed in Canadian Patent Applications 2,251,762; 2,062,214; 2,227,349; Canadian Patent 1,238,993; and PCT/CA97/00244.

Other approaches for addressing undesirable spinal curvature include the use of foot rests such as that taught in Canadian Patent Application 2,237,969 of Messinbird. As well, the use of heating pads applied to sore regions of the back or neck to reduce discomfort are common, and one example can be found in PCT/US97/23411.

Some individuals are known to suffer from a functional difference in leg length, which can place strain on various muscles and skeletal regions. While the application of massage to the affected areas has been attempted, such approaches may require frequent visits to health professionals, and results have in many cases been less than satisfactory.

Various techniques of chiropractic treatment utilize leg imbalance as a test or physical finding indicator for guiding treatment protocol. These include Activator Methods, Thompson Technique, and Grostic Cervical Technique.

Thus, despite the range of pillows and other treatments available, lower back and neck pain remains a major limitation on the quality of life enjoyed by many individuals. It is, therefore, desirable to seek alternative methods to address pain resulting from undesired musculo-skeletal imbalance.

Thus, it is an object of the invention to provide an improved method of alleviating or reducing the symptoms of undesired or musculo-skeletal imbalance.

### SUMMARY OF THE INVENTION

In an embodiment of the invention there is provided a method of alleviating or reducing the symptoms of uneven pelvic rotation in a patient suffering therefrom. The method comprises: (a) selecting the patient; (b) identifying the patient's functionally short leg; (c) positioning the patient in a substantially prone position, thereby reducing any weight supported by the patient's pelvis; (d) while the patient is in the substantially prone position, simultaneously elevating a front portion of the patient's cephalic thigh adjacent the forwardly rotated acetabular joint and removing restrictions to the rearward rotation of said acetabular joint, while elevating a cephalic portion of the front region of the ilium on the other side; and, (e) repeating step (d) at least once per month. In another embodiment of the invention there is provided the method described above wherein in step (d), pressure is applied to the cephalic portion of the ilium on the other side by placing a second therapeutic form between a front region of the cephalic portion of the ilium on the other side and the support surface.

In an embodiment of the invention there is provided a method of relieving or alleviating the symptoms of chronic spinal movement (or misalignment) in the neck in a patient suffering therefrom. The method comprises: (a) selecting the patient; (b) identifying the direction of spinal movement (or misalignment) in the patient's neck; (c) positioning the patient in a substantially horizontal position looking upward so as to reduce any weight supported by the patient's neck; and, (d) applying a consistent force to the back of the patient's neck in the region of the second to fourth cervical vertebrae, thereby urging lateral displacement of the cervical spine in a direction opposite to the direction of spinal movement (or misalignment) in the patient's neck.

In an embodiment of the invention there is provided a therapeutic form comprising a flexible outer surface and an inner filling contained within the outer surface. The inner filling includes a soft filling and a firm filling which is less compressible than the soft filling. The outer surface and filling are shaped so as to result when combined in an elongate structure having a long axis, a first end and a second end. The soft filling occupies a region within said outer surface extending from the first end along at least half of the length of the long axis toward the second end. The firm filling occupies a region within the outer surface extending from the second end along the remainder of the length of the long axis up to the region occupied by the soft filling, such that in operation a patient who places his neck on said roll will find it urged to move toward the direction of the soft filling.

In an embodiment of the invention there is provided a method of identifying the direction of spinal movement in the neck of a patient suffering from uneven pelvic rotation and movement of the cervical spine. The method comprises the following sequential steps: (a) selecting the patient; (b) identifying the patient's rearwardly rotated ilium; (c) applying finger pressure to one side of the patient's neck adjacent the cervical vertebrae; (d) examining the patient's rearwardly rotated ilium (functional leg length) to determine if the rotation has increased, decreased or remained the same; (e) if the rotation has increased or remained the same, repeating step (c) on the other side of the patient's neck and re-examining the patient's rearwardly rotated ilium (functional leg length) to determine if the forward rotation has increased, decreased or remained the same; and, (f) if the rotation has decreased, observing that the side of the neck to which pressure was applied according to step c) immediately preceding the decrease in rotation is the direction towards which the patient's spine in the neck is moved.

In an embodiment of the invention there is provided a collapsible therapeutic wedge comprising: a bottom plate having a bottom plate edge and a strut region; a top plate having a top plate edge and a securing region; and, a support having a top end and a bottom end. The top plate edge is rotatably secured to said bottom plate edge and is rotatable between an open position and a closed position. The support top end is adapted to engage the securing region and the support bottom end is adapted to engage the strut region, such that in operation said strut can retain said top plate in the open position.

### BRIEF DESCRIPTION OF THE DRAWINGS

These and other advantages of the invention will become apparent upon reading the following detailed description and upon referring to the drawings in which:—

FIG. 1a is a depiction of a human pelvic bone showing certain possible rotations thereof.

FIG. 1b is a depiction of a human subject suffering from a form of function leg length differential related to unbalanced pelvic rotation.

FIG. 2a is a top view of a portion of a patient employing therapeutic forms to alleviate or reduce the symptoms of unbalanced pelvic rotation.

FIG. 2b is a left side view of the patient of FIG. 2a.

FIG. 3 is a perspective view of an embodiment of two therapeutic wedges and an embodiment of a neck roll of the present invention.

FIG. 4 is a side view of a portion of a patient employing an embodiment of the neck roll of the present invention.

FIG. 5 is a perspective view of an embodiment of the neck roll of the present invention.

FIGS. 6a and b provide perspective views of an embodiment of a collapsible therapeutic wedge of the present invention.

While the invention will be described in conjunction with the illustrated embodiments, it will be understood that it is not intended to limit the invention to such embodiments. On the contrary, it is intended to cover all alternatives, modifications and equivalents as may be included within the spirit and scope of the invention as defined by the appended claims.

### DETAILED DESCRIPTION OF THE INVENTION

In the following description, similar features in the drawings have been given similar reference numerals.

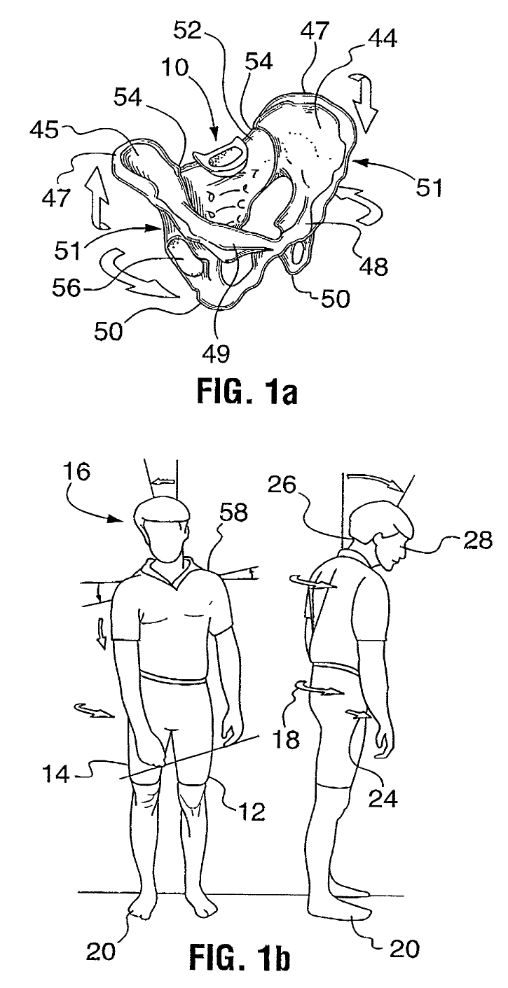

FIG. 1a depicts a human pelvic bone 10, together with arrows showing the direction of rotation of the pelvis which one would expect to see in a human patient having a long and short functional leg length differential, wherein the left leg 12 (attached to the distant side of the pelvis in FIG. 1a) would be functionally shorter than the right leg 14.

As shown in FIG. 1a, the human pelvis 10 includes left and right innominatum bones 51. The innominatum bones 51 are joined to opposite sides of the sacrum 52.

Each innominatum bone 51 includes an ilium 45, a pubic bone 49 and an ischium 50. The top edge of the ilium 45 is referred to as the iliac crest 47.

Each innominatum bone 51 is capable of some rotation relative to the sacrum 52 by movement about the sacro-iliac joint 54.

The legs are joined to the pelvis 10 at the acetabular joint 56.

Pelvic imbalance (or innominate rotation), resulting in one leg having a functional length greater than the other, is quite common, and frequently results from the undesirable rotation of the innominatum bone 51 on one side of the pelvis forward about the sacro-iliac joint 54 to move the pubic bone 49 and acetabular joint 56 forward on that side, which results in the corresponding leg being functionally shorter. This is often found together with elevation of the shoulder 58 on the short leg side. The arrows in FIG. 1a show rotation consistent with a functional left-leg-short condition.

The undesirable rotation of an innominatum bone 51 of the pelvis 10 moves the acetabular joint 56 (acetabular region) forward and the ilium 45 on the same side backward. The side with the forwardly rotated acetabular joint (rearwardly rotated ilium) is the short leg side. In general, the innominatum bone 51 on the other (long leg) side is rotated so that the ilium 45 on that side is moved forward and the acetabular joint 56 is moved backward.

FIG. 1b shows a human subject 16 suffering from pelvic rotation, wherein the left acetabular joint 56 is rotated forward, causing the left leg 12 to be functionally shorter than the right leg 14. As shown by the arrows in FIG. 1b, numerous secondary adjustments made by the muscles and the spine frequently compensate for such pelvic imbalance, causing movement of the skeleton above the pelvis and, frequently, a twisting imbalance (twisting) of the cervical spine.

In one aspect of the invention, there is provided a method of alleviating or reducing the symptoms of pelvic imbalance in a patient. Preferably, the rearwardly rotated ilium is gently urged back into a more neutral position, while the other side of the pelvis, which is frequently slightly forward rotated, is permitted to rotate rearward, also into a more neutral position.

While it is not intended to restrict the invention to any particular mechanism, and it will be understood that the invention is limited only by the claims appended hereto, it is believed that returning the innominatum bones 51 and acetabulate joints 56 to a more neutral rotation relative to each other, such that legs have approximately the same functional length will, in an ordinary individual, trigger the relaxation of various muscles in the back and neck which ordinarily compensate for the uneven pelvic rotation.

As used herein, the terms “pelvic rotation” and “pelvic imbalance” refer to movement of a human innominatum bone 51 about an axis which, when the patient is standing upright, is substantially horizontal, and extends to the patient's sides, at right angles to the patient's direction of forward vision. As used herein, the term “rearwardly rotated ilium” refers to an innominatum bone 51 which is rotated about the above-described axis such that the acetabular joint 56 is rotated toward the front of the individual, and generally upwardly. In such cases, the ilium 45 on that side is rotated toward the back of the individual.

As used herein, the term “forward” when used to identify a region of a limb or body part refers to that portion of the limb or body part which is located closer to the portion of the individual which faces forward when that individual is standing in a normal upright position. As used herein, the term “rearward” when used to identify a region of a limb or body part refers to that portion of the limb or other body part which is located closest to the back of an individual when that individual is standing in a normal upright position.

As used herein, the term “cephalic” refers to that portion of a limb or other body part located closest to the individual's head, when that individual is in an upright standing position. As used herein, the term “caudal” refers to that portion of a limb or other body part closest to that individual's feet when the individual is standing in a normal upright position.

As used herein, the term “he” (and “his”) includes “she” (and “hers”) and vice-versa.

In one embodiment of the invention there is provided a method to effect a long-term reduction in pelvic imbalance comprising applying the method of alleviating or reducing the symptoms of pelvic imbalance on a regular basis. Preferably the method is applied at least once per week, and more preferably at least twice per week.

In an embodiment of the invention there is provided a method for substantially eliminating muscular pain associated with chronic pelvic imbalance comprising applying the method of alleviating or reducing the symptoms of pelvic imbalance between once per month and once every two weeks, more preferably between once every two to three weeks.

### EXAMPLE 1

### Pelvic Imbalance

### Example 1A

### Identifying the Rearwardly Rotated Ilium

In one embodiment of the method of the present invention, uneven pelvic rotation in a patient can be identified by identifying a functional leg length differential which is indicative of the forward rotation of one acetabular joint.

The forwardly rotated acetabular joint can be readily determined by placing the patient in a generally prone position on a support surface 21, so as to substantially remove any load from the legs and pelvis while preferably supporting the patient's body from the head to somewhat below the knees. The patient's feet 20 should be allowed to dangle freely off of the end of the support surface, and the patient's head should preferably be supported such that the face 28 remains in a comfortable downwardly-looking position, without unnaturally twisting or kinking the spine. It is particularly preferred to use a massage table for this purpose, although any suitable horizontal support may be employed.

In order to identify the forwardly rotated acetabular joint (and the related rearwardly rotated ilium) of a patient from the prone position, the length of the legs is compared, by looking at the relative position of the heels of the patient as they dangle off the support surface 21. For a patient with a functional leg length differential, the leg which appears shortest, when dangling off the edge of the surface, is attached to the forwardly rotated acetabular region on the same side as the rearwardly rotated iliac region.

In some instances it will be desirable to confirm the assessment of the forwardly rotated acetabular region, by instructing the patient to relax and then gently raising the feet, allowing the knees to bend, until the soles of the feet are substantially parallel to the ceiling, and then allowing the feet to gently and slowly fall back to the substantially horizontal position. In many instances, this will aggravate the leg length differential, making it easier to identify the functionally short leg, and therefore the forwardly rotated acetabular and rearwardly rotated ilium.

In some instances it will be desirable to confirm which ilium is rearwardly rotated by gently urging the ilium which is believed to be rearwardly rotated towards a more neutral position. This may be accomplished by gently but firmly massaging the top of the gluteus maximus in a caudal to cephalic direction for several moments. In some instances it will be desirable to apply gentle frontly-directed pressure against a rearward portion of the iliac crest. When the rearwardly rotated ilium has been correctly assessed, such massage will in many instances trigger a relaxation of the pelvis into a more neutral position, which will result in a reduction of the functional leg length differential. In any event, if this massaging increases the functional leg length differential, the assessment of the forwardly rotated acetabular joint and rearwardly rotated ilium may have been erroneous and should be repeated.

### Example 1B

### Reducing Pelvic Imbalance

In order to alleviate or reduce the on-going symptoms of pelvic imbalance, and particularly those symptoms relating to soft tissue and muscle stress and spinal imbalance which results to compensate for uneven pelvic rotation and to promote a long-term reduction in pelvic imbalance, it is desirable to urge the pelvis into a neutral configuration on a regular basis, and preferably at least once per week. Once the patient's rearwardly rotated ilium (and forwardly rotated acetabular joint) has been identified, and the patient is in a substantially prone position with the spine straight and the neck comfortable, as described above, therapeutic forms may be employed to urge the pelvis into a more neutral orientation.

The forwardly rotated acetabular joint associated with the functionally short leg may be urged into a more neutral position by placing a therapeutic form under the patient's thigh adjacent to the forwardly rotated acetabular joint. Preferably, the therapeutic form is applied against a front portion of the patient's cephalic thigh 24, such that the thigh is elevated above the support surface, causing the force of gravity to rotate the pelvis downward relative to the thigh and toward the support surface, and thereby causing the acetabular joint to rotate away from the forward position.

The therapeutic form employed may be any suitable object capable of supporting the weight of the patient's thigh, when the patient is in a prone position, and elevating the thigh between about one and about six inches above the support surface. Preferably, the therapeutic form is adapted to elevate the thigh between 2 and 4 inches above the support surface 21. In some instances the therapeutic form may be made from a slightly compressible material, to enhance patient comfort, without being so compressible as to fail to elevate the thigh appropriately.

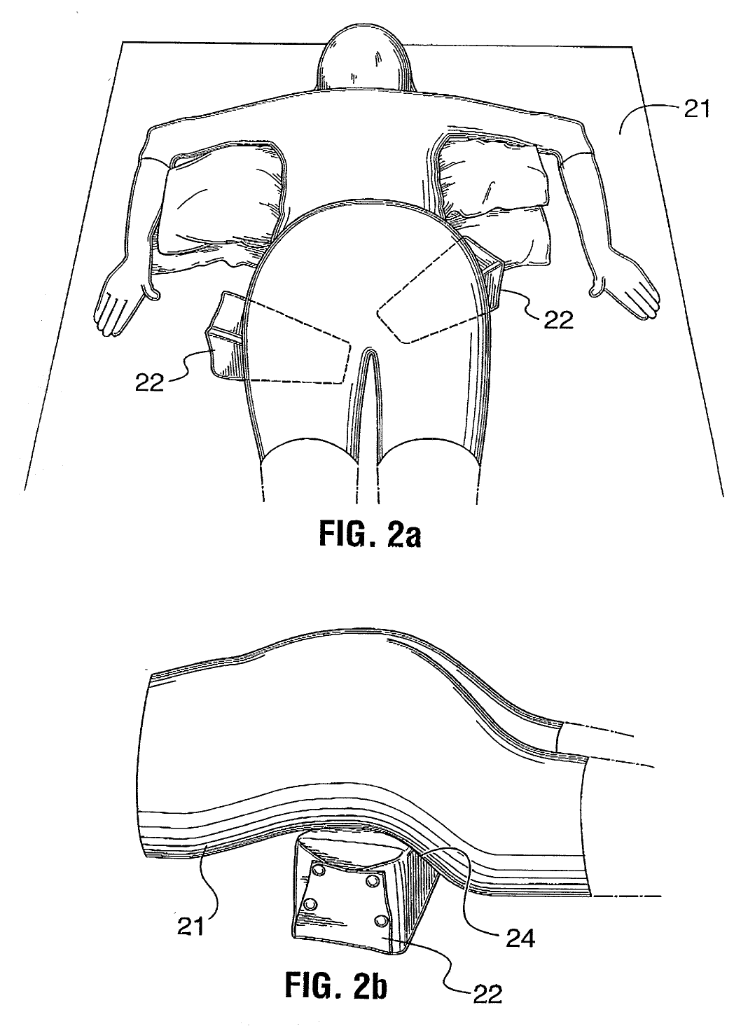

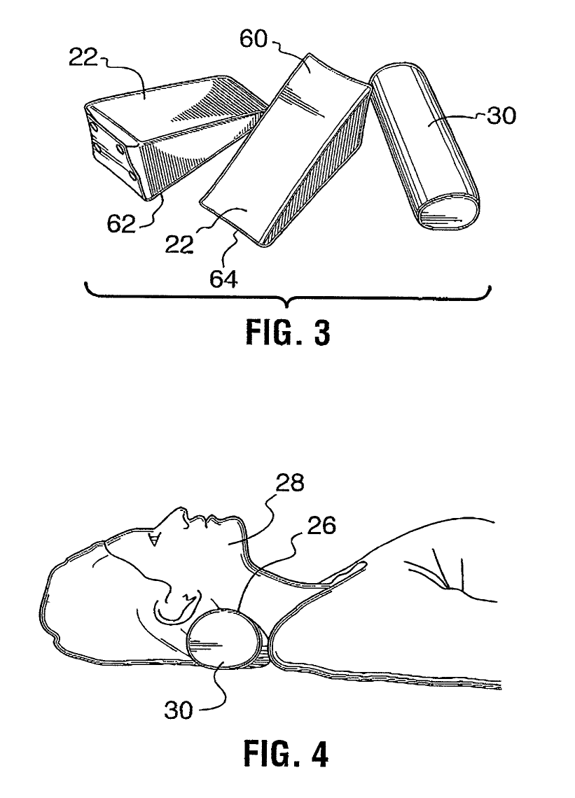

The therapeutic form is of a shape which allows its insertion under the thigh, without necessitating contact between the form and the pelvis itself. In some instances, it will be desirable to use a wedge-shaped therapeutic form of the type herein referred to as therapeutic wedges 22 and shown in use in FIGS. 2a and 2b, as well as in FIG. 3 (on the left hand side). Wedge-shaped forms have an advantage of allowing ready adjustment of the extent of elevation of the thigh above the support surface. However, it will be understood that any suitable object can be employed, including a rolled up towel, a small firm pillow, or other objects of sufficient thickness and resilience such as one or more heating pads.

While urging the patient's forwardly rotated acetabular joint into a more neutral position, it will often be desirable to facilitate rotation of the patient's other innominatum bone toward a more neutral position. This can be accomplished by placing a second therapeutic form against an cephalic portion of the front region of the other ilium. In some instances, this pressure is preferably applied to the area around where one would find the belt line in a typical male patient (although the patient may be male or female). In some instances the pressure is preferably applied at the cephalic superior iliac spine.

In some instances it will be desirable to raise the patient's cephalic thigh off the support to a height which is less than the height which the cephalic portion of the patient's other innominatum bone is raised off the support.

Examples of use of an embodiment of a therapeutic form to carry out the method of alleviating or reducing the symptoms of uneven pelvic rotation in a patient is shown in FIGS. 2a and 2b. In these figures, the patient was treated for functional leg length imbalance wherein the left leg was functionally shorter than the right. FIG. 2b shows in more detail the positioning of the therapeutic wedge under the cephalic thigh adjacent the forwardly rotated acetabular joint.

### EXAMPLE 2

### Imbalance of the Cervical Spine

### Example 2A

### Identifying the Direction of Cervical Spine Imbalance

As discussed above, pelvic imbalance can often lead to an imbalance of the spine above the pelvis, and particularly abnormal lateral movement or misalignment of the apex of curvature of the cervical spine. Such movement, and the pain associated with it, can be alleviated using the correct application of suitable therapeutic forms. In some instances, however, it is desirable to identify the direction of spinal movement in the neck 26 of the patient.

In order to identify the direction of movement of the cervical spine of a patient having uneven pelvic rotation, the patient is preferably placed in a generally prone position and supported at least from the head to somewhat below the knees, with the feet 20 hanging off the end of the support surface 21, the face 28 directed down in a comfortable manner, and with the spine being substantially straight. A massage table is suitable for this purpose, although a variety of other apparatus may be employed, and in fact the patient may simply be suspended along a series of strategically placed pillows or cushions.

The patient's forwardly functionally short leg is identified, for example as described in Example 1a. Following identification of the functionally short leg, finger pressure is applied gently but firmly to one side of the patient's neck adjacent the cervical vertebrae. Following application of finger pressure, and particularly finger pressure adjacent cervical vertebrae two through six, the extent of the patient's pelvic imbalance is examined by determining if the functional leg length differential has increased, stayed the same, or decreased.

If the functional leg length differential has remained the same or has increased, finger pressure should then be applied firmly but gently to the other side of the patient's neck adjacent the opposite side of the same cervical vertebrae, and the extent of the functional leg length differential should once again be assessed.

If the extent of the differential has decreased, one can conclude that the spinal movement in the neck of the patient is directed toward the side of the patient's neck to which pressure was most recently applied.

Alternatively or additionally, the direction of cervical spine movement can be determined by an assessment of neck muscle tension in the patient. The side of the neck which the muscle is less tight is ordinarily the side towards which the apex of the curvature of the cervical spine has moved.

### Example 2B

### Reducing Undesired Movement or Misalignment of the Cervical Spine

In some instances it will be desirable to relieve or alleviate the symptoms of chronic spinal movement (or misalignment) in the neck. Such a method may be carried out by selecting a patient suffering from chronic spinal movement (or misalignment) in the neck, identifying the direction of spinal movement in the patient's neck, and placing the patient in a substantially horizontal position looking upward with the spine generally laterally straight. A consistent force is then applied to the back of the patient's neck. This force gently urges movement of the neck (and particularly the apex of the curve of the cervical spine) in a direction opposite to the direction of spinal movement.

Preferably, the consistent force is applied to the back of the patient's neck in the region of cervical vertebrae two through four. Preferably, movement of the neck of between 0.1 and 5 cm is urged. It will be apparent to those skilled in the art that the full extent of desirable movement of the neck will depend on many factors, including the patient's overall condition, previous neck injuries, the extent of mobility, and the duration for which rotation is to be maintained. In some instances it will be desirable to maintain the urged movement of the neck for between about several minutes to eight hours. In other instances, it will be desirable to maintain the urged movement between about five minutes and one hour.

A suitable therapeutic form can be used to apply the desired consistent force to the back of the patient's neck. In particular, a therapeutic form shaped to fit within and support the natural healthy cervical curve of the neck can be employed. The therapeutic form preferably applies greater pressure to one side of the neck than the other, with greater pressure being applied on the side toward which the spine has undesirably moved. The uneven force generated by the therapeutic form may be provided through the use of a filling of differing densities in the therapeutic form, such that the portion of the neck resting on the softer filling is less well supported, causing the neck to naturally tend toward that direction. Alternatively, the form itself may be shaped, for example in a taper, such that the form applies greater pressure to one side of the neck than the other. In some instances it will be desirable to use a therapeutic form which causes the patient's cervical spine to arch to form a forwardly convex curve between about the odontoid process and the second dorsal vertebrae.

While the method has been described in respect of a patient lying in a generally horizontal position while looking upward, it will be understood that the method can also be conducted with the patient in differing positions, so long as the spine is correctly supported and aligned, and the neck is appropriately moved.

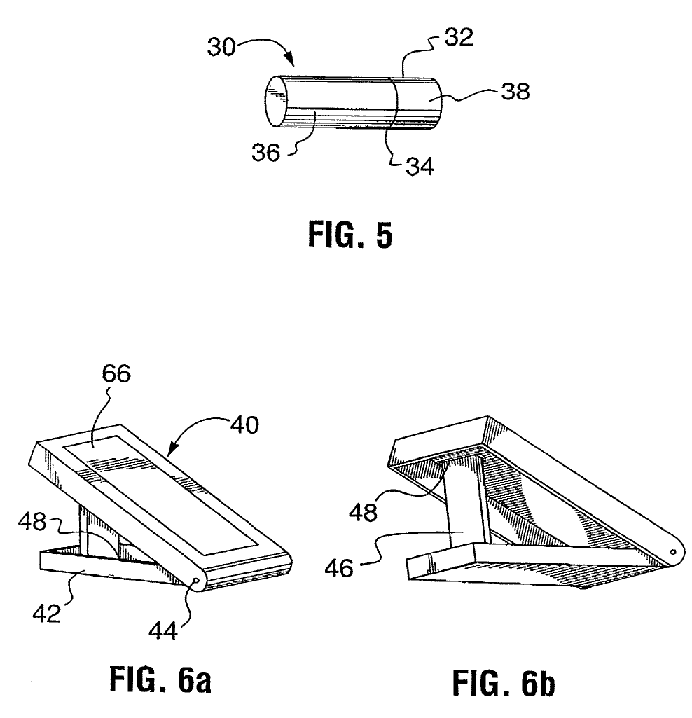

In one embodiment of the invention there is provided a therapeutic form suitable for use in the method of relieving or alleviating the symptoms of chronic spinal movement which is a neck roll 30. As shown in FIG. 5, the neck roll 30 may comprise a flexible outer surface 32, such as an integral “skin” or a cloth or plastic “pillow case”, surrounding an inner filling 34. The inner filling 34 includes a soft filling 36 and a firm filling 38, with the firm filling 38 being less compressible than the soft filling 36. The flexible structure 32, when filled with the inner filling 34, results in an elongate structure having a long axis, and two ends. The soft filling preferably occupies at least one half of the length of the structure, whereas the firm filling occupies the remaining length. Thus, in one embodiment, the elongate structure is filled for two thirds of its length with a soft filling, and for the final third with a firm filling. Thus, a patient who places the elongate structure under their neck or another region of their body (with the firm filled region under one side of the neck or body), will find that the elongate structure urges movement towards the side filled with the soft filling.

Where the therapeutic form is to be used under the neck, it will often be preferable that the soft filling portion of the form have a width when compressed that is no greater than a normal healthy comfortable cervical arch. In some instances, it will be desirable for the elongate structure to be a roll shape, in which case the therapeutic form, when sized appropriately for use with the neck, may be referred to as a neck roll.

The therapeutic form for use under the neck is preferably between about 6 and 16 inches long and between about 1 and 4 inches wide, when compressed by the weight of an average patient's neck.

One embodiment of a neck roll 30 is depicted in FIG. 3 (at the far right). FIG. 4 depicts an embodiment of a neck roll in operation under a patient's neck.

FIG. 5 depicts an embodiment of a neck roll 30 having a flexible outer surface 32 and an inner filling 34, wherein the inner filling includes a soft filling 36 and a firm filling 38.

It will be appreciated that the outer surface need not be structurally distinct from the inner filling, and may in fact be a polymer “skin” formed on foam used to produce the therapeutic form, such as integral-skin on polyurethane foam.

For example a neck roll having a total length of 9 inches can be made by joining a 6¾ inch soft filling region and a 2¼ inch firm filling region. By way of example the soft filling can have a 75% ILD (“indentation, load, and deflection”) ASPM rating of 60 pounds and the firm filling can have a 75% ILD ASPM rating of 100 pounds. (75% ILD (ASPM) is determined by taking a 15″(l)×15″(w)×4″(h) sized sample, and placing an 8″ diameter disc on the center of it. The disc weight (lbs) which causes the sample height to reduce from 4″ to 3″ is the 75% ILD ASPM weight.)

In some instances it will be desirable to use a soft filling having a 75% ILD ASPM rating of between about 40 and 80 pounds and a firm filling having a 75% ILD ASPM rating of between about 80 and 120 pounds. It will be appreciated that the preferred filling density can be determined in light of the disclosure herein and the patient's size, weight and condition.

For certain patients polyurethane foam has been employed as the neck roll filling. FOAMEX™ 145-60 has been employed as soft filling and FOAMEX™ 22-100 has been employed as firm filling.

Alternatively or additionally, the therapeutic form may be inflatable or water filled, such that the soft and firm fillings are created using two separate compartments within the outer surface 32, whereby the “soft filling” side is filled to a lower pressure than is the “firm filling” side.

Moreover, in some instances, it will be desirable to employ therapeutic forms which are adapted to be collapsible for ease of transport and use by travelers.

Therapeutic wedges preferably include a top surface and an aligned bottom surface, a support separating first ends of the surfaces, and a join region connecting second ends of the surfaces. In some instances the top surface may include a comfort coating which may be soft or compressible. In some instances the bottom surface may include an antislip coating.

FIG. 6 depicts an alternative embodiment of the therapeutic wedge 22 adapted to be collapsible when needed. This collapsible wedge includes a top plate 40, a bottom plate 42, a hinge 44, a support 46 and support securers 48 adapted to releasably secure the support 46 to the top plate 40 and the bottom plate 42. In some instances, the support securer connecting the support 46 to the top plate 40 is a support hinge and the support securer connecting the support 46 to the bottom plate 42 is a snap.

In some instances it will be desirable to have a kit containing at least 2 or 3 therapeutic forms which might include 1 or 2 therapeutic wedges optionally combined with a neck roll, together with instructions for their use.

Thus, it is apparent that there has been provided in accordance with the invention a method for reducing musculo-skeletal imbalance that fully satisfies the objects, aims and advantages set forth above. While the invention has been described in conjunction with specific embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

What I claim as my invention:

1. A method of alleviating or reducing the symptoms of uneven pelvic rotation in a patient suffering therefrom, comprising:
(a) selecting the patient;
(b) identifying the patient's functionally short leg;
(c) positioning the patient in a substantially prone position, thereby reducing any weight supported by the patient's pelvis;
(d) while the patient is in the substantially prone position, simultaneously elevating a front portion of the patient's cephalic thigh adjacent the forwardly rotated acetabular joint and removing restrictions to the rearward rotation of said acetabular joint, while elevating a cephalic portion of the front region of the ilium on the other side; and
(e) repeating step (d) at least once per month.

2. The method of claim 1 wherein in step (d), pressure is applied to the cephalic portion of the patient's thigh by positioning a first therapeutic form between the upper thigh and the support surface.

3. The method of claim 2 wherein in step (d), pressure is applied to the cephalic portion of the ilium on the other side by placing a second therapeutic form between a front region of the cephalic portion of the ilium on the other side and the support surface.

4. The method of claim 3 wherein said first therapeutic form is positioned so as to space the patient's cephalic thigh off the support by an amount smaller than the amount which the cephalic portion of the ilium on the other side is spaced off the support by said second therapeutic form.

5. The method of claim 3 wherein the therapeutic form is a therapeutic wedge.

6. The method of claim 3 wherein pressure is applied to the cephalic portion of the front region of the patient's ilium at about the belt line.

7. The method of claim 3 wherein pressure is applied to the cephalic portion of the front region of the patient's ilium at the cephalic superior iliac spine.

8. A method of relieving or alleviating the symptoms of chronic spinal movement in the neck in a patient suffering therefrom, comprising:
(a) selecting the patient;
(b) identifying the direction of spinal movement in the patient's neck;
(c) positioning the patient in a substantially horizontal position looking upward so as to reduce any weight supported by the patient's neck; and,
(d) applying a consistent force to the back of the patient's neck in the region of the second to fourth cervical vertebrae, thereby urging lateral displacement of the cervical spine in a direction opposite to the direction of spinal movement in the patient's neck.

9. The method of claim 8 wherein the patient's cervical spine is further arched to form a forwardly convex curve between about the odontoid process and the second dorsal vertebra.

10. The method of claim 9 wherein in step (c) the patient is positioned substantially lying on his or her back against a support surface.

11. The method of claim 10 wherein in step (d) the force is applied by a therapeutic form placed between the back of the patient's neck and the support surface.

12. The method of claim 11 wherein the therapeutic form is a neck roll.

13. A therapeutic form comprising:
a flexible outer surface;
an inner filling contained within said outer surface;
said inner filling including a soft filling and a firm filling which is less compressible than said soft filling;
said outer surface and filling being shaped so as to result when combined in an elongate structure having a long axis, a first end and a second end;
said soft filling occupying a region within said outer surface extending from said first end along at least half of the length of the long axis toward said second end;
said firm filling occupying a region within said outer surface extending from said second end along the remainder of the length of the long axis up to the region occupied by said soft filling;
such that in operation a patient who places his neck on said roll will find it urged to move toward the direction of the soft filling.

14. The method of claim 11 wherein the therapeutic form is the therapeutic form of claim 13.

15. A method of identifying the direction of spinal movement in the neck of a patient suffering from uneven pelvic rotation and movement of the cervical spine, comprising the following sequential steps:
(a) selecting the patient;
(b) identifying the patient's rearwardly rotated ilium;
(c) applying finger pressure to one side of the patient's neck adjacent the cervical vertebrae;
(d) examining the patient's rearwardly rotated ilium to determine if the rotation has increased, decreased or remained the same;
(e) if the rotation has increased or remained the same, repeating step (c) on the other side of the patient's neck and re-examining the patient's rearwardly rotated ilium to determine if the forward rotation has increased, decreased or remained the same; and,
(f) if the rotation has decreased, observing that the side of the neck to which pressure was applied according to step c) immediately preceding the decrease in rotation is the direction towards which the patient's cervical spine is moved.

16. The method of claim 8 wherein step (b) is conducted according to the method of claim 15.

17. A collapsible therapeutic wedge comprising:
a bottom plate having a bottom plate edge and a strut region;
a top plate having a top plate edge and a securing region;
said top plate edge being rotatably secured to said bottom plate edge and being rotatable between an open position and a closed position;
a support having a top end and a bottom end; and,
said support top end being adapted to engage said securing region and said support bottom end being adapted to engage said strut region, such that in operation said strut can retain said top plate in the open position.

18. A kit comprising:
(a) two therapeutic wedges each including a top surface, a bottom surface, a support separating first ends of said surfaces, and a join region connecting second ends of said surfaces; and
(b) instructions for their use according to the method of claim 3.

19. The kit of claim 18 further including a neck roll.
